(19) United States
(12) Patent Application Publication
(10) Pub. No.: US 2026/0215714 A1
(43) Pub. Date: Jul. 30, 2026

(54) URINARY PRESSURE MEASUREMENT METHOD AND DEVICE USING IMAGES

(71) Applicant: GANGNEUNG-WONJU NATIONAL UNIVERSITY INDUSTRY ACADEMY COOPERATION GROUP, Gangneung-si, Gangwon-do (KR)

(72) Inventor: Jae Min BAEK, Wonju-si, Gangwon-do (KR)

(21) Appl. No.: 19/146,197

(22) PCT Filed: Dec. 15, 2023
(86) PCT No.: PCT/KR2023/020712

§ 371 Date: Jul. 18, 2025

(30) Foreign Application Priority Data
Jan. 5, 2023 (KR) 10-2023-0001582

(51) Int. Cl. A61B 5/20; A61B 5/00

(52) U.S. Cl. CPC A61B 5/20; A61B 5/0082; A61B 5/7278; A61B 2576/00

(57) ABSTRACT

The present disclosure relates to a method and device of measuring urine pressure using an image, the method including: acquiring an image capturing a urine stream; obtaining a measurement variable representing a distance between two points where the urine stream is the thinnest based on the acquired image; and calculating urine pressure using the obtained measurement variable.

### TECHNICAL FIELD

The present disclosure relates to a method and device of measuring a urine pressure using an image captured by a camera.

### BACKGROUND ART

A non-invasive and relatively simple test method called uroflowmetry is used as a method to diagnose symptoms of discomfort in urination, such as prostate hypertrophy or bladder disease, which is common that collects urine during urination and measures a volume of urine (urine amount) V as a function of time t to examine a urine flow rate.

In order to conduct a uroflowmetry, first, while a subject discharges urine into a uroflowmetry collection container, the volume V and time t of urine put into the collection container are measured, and accordingly, when a time function V(t) for the volume of urine is calculated, and when the calculated function V(t) is differentiated with respect to time, a urine flow rate, which is a flow rate of urine, may be obtained.

Meanwhile, in a conventional uroflowmetry, urine discharged by a patient is accumulated in a collection container with a constant diameter, and a change in the weight of urine during the urination process is measured by a load cell under the container, and a method of obtaining the volume time function V(t) of urine based on the measured urine weight change is used.

In the case of a uroflowmetry based on the measurement of the weight of urine as described above, the weight of urine and an amount of impact generated when the urine hits the floor are measured together during the urination of the subject, and thus there is a problem in that the urine flow rate cannot be accurately measured.

### DISCLOSURE OF INVENTION

### Technical Problem

The present disclosure is proposed in consideration of the above-described conventional circumstances, and an aspect of the present disclosure is to provide a method and device of measuring urine pressure using an image so as to improve accuracy and convenience.

### Technical Solution

In order to achieve the foregoing objectives, a urine pressure measurement method according to preferred embodiments of the present disclosure, which is a method of measuring urine pressure using an image captured by a camera, may include acquiring an image capturing a urine stream; obtaining a measurement variable representing a distance between two points where the urine stream is the thinnest based on the acquired image; and calculating urine pressure using the obtained measurement variable.

The obtaining of the measurement variable may include detecting a first point and a second point where the urine stream is the thinnest in the acquired image; calculating a horizontal distance between the first and second points and a drop between the first and second points; and computing the measurement variable using the calculated horizontal distance and drop.

In addition, the obtaining of the measurement variable may include detecting, in the acquired image, a first point where the urine stream is the thinnest and a third point where the urine stream is the thickest; calculating a distance between the detected first and third points; and computing the measurement variable using the calculated distance between the first and third points.

Meanwhile, the calculating of the urine pressure may compute the urine pressure by multiplying the obtained measurement variable by a specific gravity of the urine, and an acceleration of gravity.

A urine pressure measurement device according to preferred embodiments of the present disclosure may perform a urine pressure measurement method using the image.

A urine pressure measurement device using an image may include an image acquisition unit for acquiring an image capturing a urine stream; and a urine pressure calculation unit for obtaining a measurement variable representing a distance between two points where the urine stream is the thinnest based on the acquired image, and calculating urinary pressure using the obtained measurement variable.

The image acquisition unit may include a camera mounted at a specific location to capture an image in a direction perpendicular to a direction of the urine stream, wherein the camera is mounted at a location spaced apart by a specific distance from a subject so as to allow at least one cycle having two ends at both ends where the urine stream is the thinnest is present in the image.

Meanwhile, at least some steps of the urine pressure measurement method using the image may be implemented as a computer-readable recording medium in which a program for executing a computer is recorded, and may be provided as the program itself.

### Advantageous Effects

According to an embodiment of the present disclosure, a measurement variable representing a distance between two points where a urine stream is the thinnest based on an image captured by a camera or the like may be obtained and used as a parameter for measuring urine pressure, thereby improving the convenience of urine pressure measurement.

### BRIEF DESCRIPTION OF THE DRAWINGS

FIG. 1 is a block diagram showing a configuration of a urine pressure measurement device using an image according to one embodiment of the present disclosure.

FIG. 2 is a flowchart showing one embodiment of a urine pressure measurement method using an image according to the present disclosure.

FIGS. 3 and 4 are drawings for explaining one embodiment of an image capturing a urine stream.

FIGS. 5 to 8 are drawings for explaining embodiments of a method of calculating urine pressure using a measurement variable according to the present disclosure.

### BEST MODE FOR CARRYING OUT THE INVENTION

Hereinafter, a method and device of measuring urine pressure using an image according to an embodiment of the present disclosure will be described in detail with reference to the accompanying drawings.

In describing the present disclosure below, a detailed description of related known configurations or functions incorporated herein will be omitted when it is determined that the detailed description thereof may unnecessarily obscure the subject matter of the present disclosure. The terms described below are terms defined in consideration of the functions in the present disclosure, and may vary depending on the intention or custom of the user or operator. Therefore, the terms will be defined throughout the description of the present disclosure.

In addition, in order to efficiently describe technical elements constituting the present disclosure, according to preferred embodiments of the present disclosure to be implemented below, functional components that are already provided in each system or functional components that are commonly provided in the technical field to which the present disclosure belongs will be omitted as much as possible, and functional components that must be additionally provided for the present disclosure will be mainly described.

Anyone skilled in the art will easily understand the functions of elements that have been conventionally used among the functional components that are omitted and not shown below, and also clearly understand the relationship between the components that have been omitted as described above and the components added for the present disclosure.

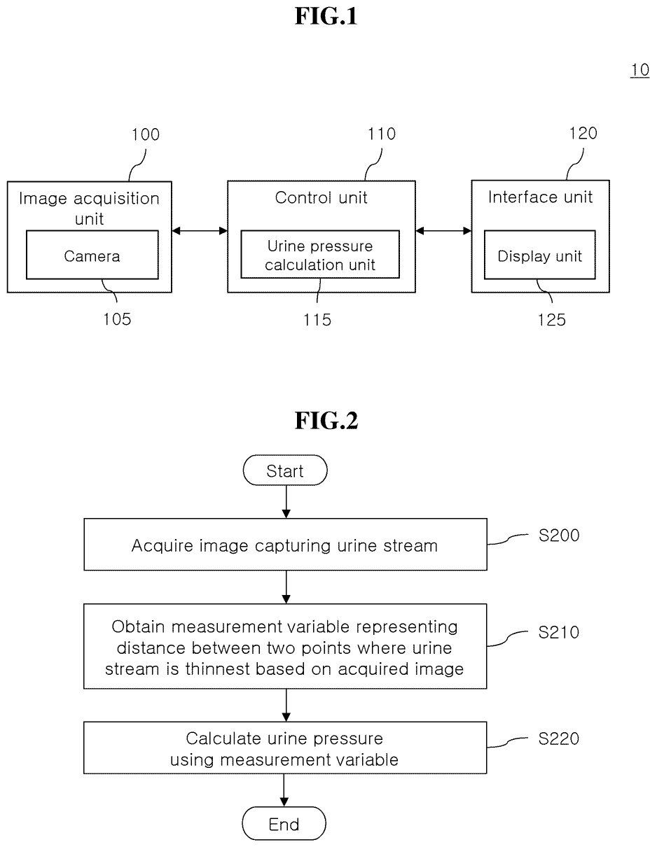

FIG. 1 is a block diagram showing a configuration of a urine pressure measurement device using an image according to one embodiment of the present disclosure, and a pressure measurement device 10 as shown above may be configured to include an image acquisition unit 100, a control unit 110, and an interface unit 120.

Referring to FIG. 1, the image acquisition unit 100 is to acquire an image capturing a urine stream discharged from a subject, and may include a camera 105 mounted at a specific location to capture an image in a direction perpendicular to a direction of the urine stream.

Meanwhile, the camera 105 may be mounted at a location spaced apart by a specific distance from the subject so as to allow at least one cycle having two points at both ends where the urine stream is the thinnest is present in the captured image.

The control unit 110 may control an overall operation of the urine pressure measurement device 10, and may include a urine pressure calculation unit 115 for obtaining a measurement variable representing a distance between two points where the urine stream is the thinnest based on an image acquired from the image acquisition unit 100, and calculating urine pressure using the obtained measurement variable.

For example, the urine pressure calculation unit 115 may detect first and second points where the urine stream is the thinnest in the image, calculate a horizontal distance between the first and second points and a drop between the first and second points, and compute a measurement variable for calculating urine pressure using the calculated horizontal distance and drop.

As another example, the urine pressure calculation unit 115 may detect a first point where the urine stream is the thinnest and a third point where the urine stream is the thickest in the image, calculate a distance between the detected first and third points, and compute a measurement variable for calculating urine pressure using the calculated distance between the first and third points.

Meanwhile, the urine pressure calculation unit 115 may compute urine pressure by multiplying the measurement variable obtained as above by a specific gravity of urine and an acceleration of gravity.

The interface unit 120 may exchange information with a user, and may include a display unit 125 for displaying urine pressure-related result values computed by the urine pressure calculation unit 115, and may include various input/output devices in addition thereto.

According to one embodiment of the present disclosure, a measurement variable representing a distance between two points where the urine stream is the thinnest based on an image captured by a camera or the like may be obtained and used as a parameter for measuring urine pressure, thereby improving the convenience of urine pressure measurement.

Hereinafter, with reference to FIGS. 2 to 8, embodiments of a urine pressure measurement method according to the present disclosure will be described in more detail.

FIG. 2 is a flowchart showing one embodiment of a urine pressure measurement method using an image according to the present disclosure, and the pressure measurement method as shown above will be described in conjunction with a block diagram showing a configuration of a urine pressure measurement device according to one embodiment of the present disclosure as shown in FIG. 1.

Referring to FIG. 2, the image acquisition unit 100 acquires an image capturing a urine stream using a camera 105 (step S200).

A urine pressure measurement method using an image according to one embodiment of the present disclosure may measure urine pressure from an image acquired by the image acquisition unit 100 based on the following four assumptions.

First, the mass does not change in the control volume, second, the cross-sectional shape of a portion where urine is discharged must be rectangular, and third, a capturing direction of the camera 105 and a direction of the urine stream must be perpendicular to each other.

For this purpose, the camera 105 can capture an image in a direction perpendicular to a direction of the urine stream, and may be mounted at a specific height at a location spaced apart by a specific distance from the subject so as to allow at least one cycle having two ends at both ends where the urine stream is the thinnest is present in the captured image.

Meanwhile, the image acquired in step S200 may include a plurality of frames captured at regular intervals from the start of urination to the end of urination, and for example, an image of 24 frames per second may be acquired, but the present disclosure is not limited thereto.

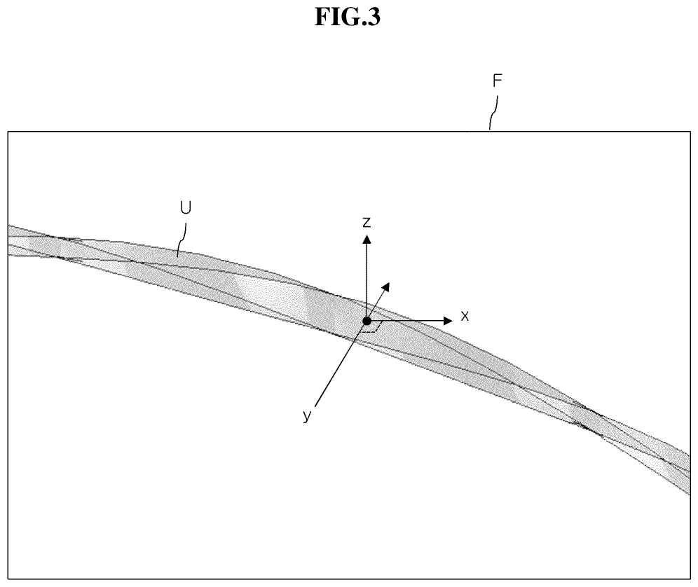

Referring to FIG. 3, one frame F of the image captured by the camera 105 may include at least part of a urine stream U discharged from a subject.

As described above, the camera 105 may be mounted at a suitable location to capture an image in a direction y perpendicular to a direction x of the urine stream U.

Additionally, as a fourth assumption, one helical cycle of the urine stream must be present in the image.

Here, one cycle of the urine stream may refer to one section having two points at both ends where the urine stream is the thinnest, and the image acquisition unit 100 may check whether at least one cycle is present in the image captured by the camera 105.

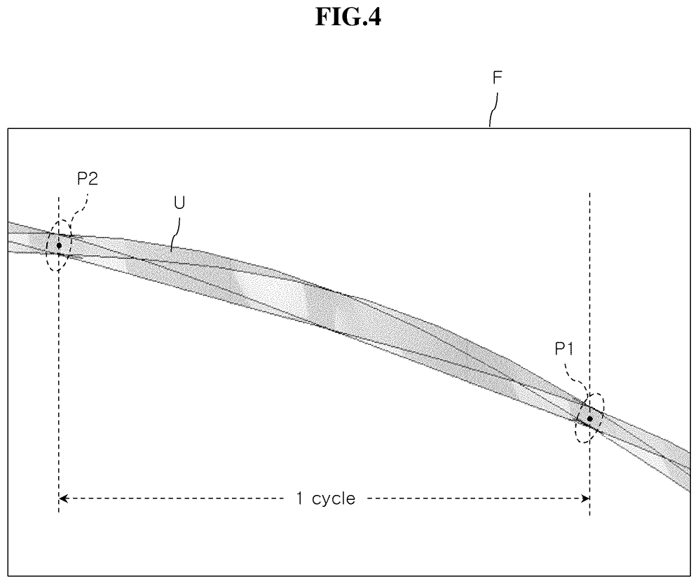

Referring to FIG. 4, points where the urine stream U is the thinnest in the image, for example, inflection points where a width of the urine stream U in the image decreases and then begins to increase, may be detected.

Then, as shown in FIG. 4, when two points P1, P2 where the urine stream U is the thinnest is present in the frame F, it may be determined that one cycle of the urine stream U is present in the frame F.

Meanwhile, when there are two or more cycles of the urine stream U in the image, a cycle located at a frontmost part of a plurality of cycles (i.e., a part where urine starts to flow) is preferably used to measure urine pressure.

In contrast, when there is no cycle of the urine stream U in the frame F, the frame cannot be used to measure urine pressure, but as the camera 105 captures an image remotely from a predetermined distance or more, one or more cycles may be present in the frame F.

The urine pressure calculation unit 115 of the control unit 110 obtains a measurement variable representing a distance between two points where the urine stream is the thinnest based on the image acquired in step S200 (step S210).

According to one embodiment of the present disclosure, a measurement variable for measuring urine pressure, which is a length of the urine stream U, may be calculated by detecting a distance between two points where the urine stream is the thinnest from the image.

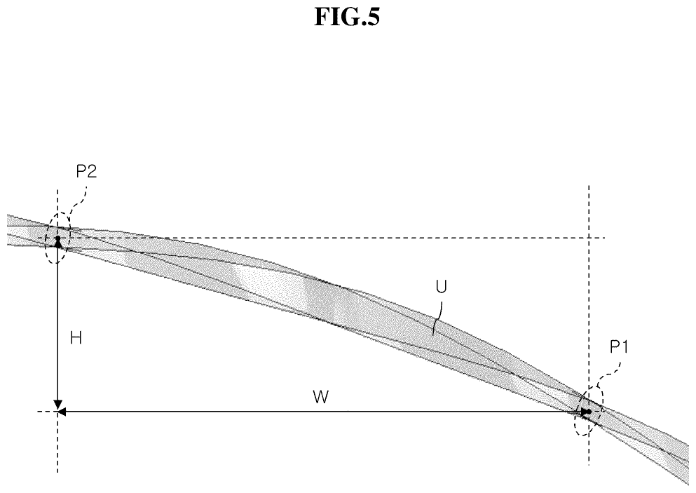

For example, referring to FIG. 5, the urine pressure calculation unit 115 may detect a first point P1 and a second point P2 where the urine stream U is the thinnest in the image, calculate a horizontal distance W between the first and second points P1, P2 and a drop H between the first and second points P1, P2, and compute a measurement variable for measuring urine pressure using the calculated horizontal distance W and drop H.

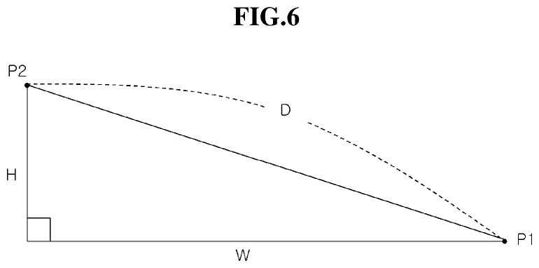

Referring to FIG. 6, a length of the urine stream U, which is a measurement variable for measuring urine pressure, may be approximated by a linear distance D between the first and second points P1, P2 where the urine stream U is the thinnest.

Meanwhile, the linear distance D between the first and second points P1, P2 may be computed by using a horizontal distance W and a drop H between the first and second points P1, P2 as in Equation 1 below.

$$D = \sqrt{{W}^{2} + {H}^{2}} \qquad [ Equation 1 ]$$

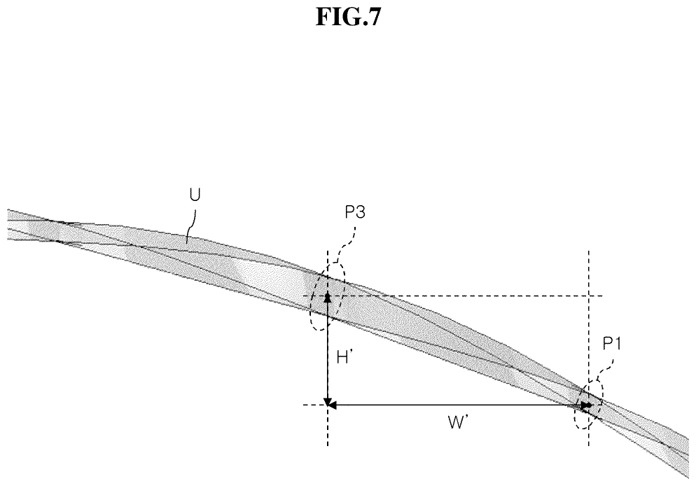

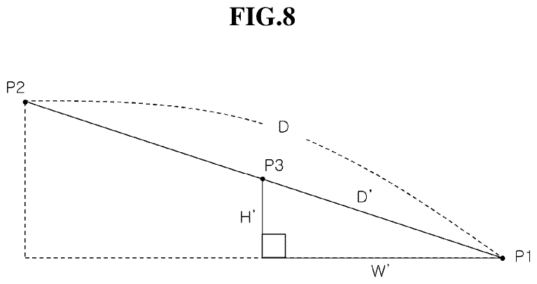

As another example, referring to FIG. 7, the urine pressure calculation unit 115 may detect a first point P1 where the urine stream U is the thinnest and a third point P3 where the urine stream U is the thickest in the image, calculate a distance between the detected first and third points P1, P3, and then compute a measurement variable for measuring urine pressure using the calculated distance between the first and third points.

Referring to FIG. 8, a length of the urine stream U, which is a measurement variable for measuring urine pressure, may be approximated as twice a linear distance D′ between the first and third points P1, P3.

Accordingly, a linear distance D between the first and second points P1, P2 may be computed by using a horizontal distance W′ and a drop H′ between the first and third points P1, P3, as in Equation 2 below.

$$D = 2 \times \sqrt{{W}^{′2} + {H}^{′2}} \qquad [ Equation 2 ]$$

Then, the urine pressure calculation unit 115 may compute urine pressure by multiplying the measurement variable obtained as above by a specific gravity of urine and an acceleration of gravity.

For example, a weight F of the urine stream U may be computed using a specific gravity p of a fluid, a volume V, and an acceleration of gravity g as in Equation 3 below.

$$F = ρ Vg \qquad [ Equation 3 ]$$

Meanwhile, the volume V of the urine stream U may be computed using a unit area A and a length D as in Equation 4 below.

$$V = AD \qquad [ Equation 4 ]$$

In addition, by substituting Equation 4 into Equation 3, a stress F of the urine stream U may be obtained as in Equation 5 below.

$$F = ρ AgD \qquad [ Equation 5 ]$$

In the above Equation 5, the unit area A may be computed as a product ab of a width a at a point where the urine stream U is the thinnest and a width b at a point where it is the thickest in the image. For example, referring to FIG. 7, the unit area A may be calculated by multiplying a width of the first point P1 where the urine stream U is the thinnest by a width of the third point P3 where the urine stream U is the thickest in the image.

Furthermore, a pressure of the urine stream U, that is, a urine pressure P, may be obtained by multiplying the stress F of the urine stream U by a constant α, as in Equation 6 below.

$$P = α F = αρ AgD \qquad [ Equation 6 ]$$

Here, the constant α, which is a proportional constant between the stress F of the urine stream U and the urine pressure P, may be set based on the stress F of the urine stream U calculated through an image and the urine pressure P precisely measured through a separate urine pressure meter according to one embodiment of the present disclosure.

For example, data on a value of the urine pressure P measured by the urine pressure meter divided by the stress F of the urine stream U calculated through an image according to one embodiment of the present disclosure may be acquired a plurality of times, and an average of the acquired data may be taken so as to determine a proportional constant α between the stress F of the urine stream U and the urine pressure P.

Meanwhile, as in the above Equation 6, a distance D between the first and second points P1, P2 representing a length of the urine stream U, which is a measurement variable for measuring urine pressure, may be multiplied by a constant α, a specific gravity p of a fluid, a unit area A, and an acceleration of gravity g, to compute the urine pressure P.

As described above, an image acquired through the image acquisition unit 100 may include a plurality of frames captured at regular intervals from the start of urination to the end of urination, and in this case, the foregoing steps S210 and S220 may be performed for each frame so as to compute the urine pressure for each of the plurality of frames.

Accordingly, the control unit 110 may calculate at least one of a graph representing a change in urine pressure, a maximum urine pressure value, and an average urine pressure value, using the computed urine pressure for each of the plurality of frames, and the calculated graph, maximum urine pressure value, average urine pressure value, and the like may be displayed through the display unit 125 of the interface unit 120.

According to another embodiment of the present disclosure, in addition to measuring urine pressure as described above, a urine amount M, which represents an amount of urine, may also be measured through an image.

For example, the urine amount M is computed by multiplying a unit area A of the urine stream U by a velocity V₀, as in Equation 7 below.

$$M = {AV}_{0} \qquad [ Equation 7 ]$$

In Equation 7, a velocity V₀ of the urine stream U may be measured using various existing methods that measure a flow velocity, and for example, may be measured using a particle image velocimetry (PIV), but the present disclosure is not limited thereto.

The foregoing methods according to one embodiment of the present disclosure may be produced as a program to be executed on a computer. In addition, the program may be stored in a computer-readable recording medium, and examples of computer-readable recording media include a ROM, a RAM, a CD-ROM, a magnetic tape, a floppy disk, an optical data storage device, and the like.

The computer-readable recording medium may be distributed over computer systems connected via a network, and stored and executed as computer-readable codes in a distributed manner. Furthermore, functional programs, codes, and code segments for implementing the method may be easily inferred by programmers in the technical field to which the present disclosure pertains.

In addition, while the preferred embodiments of the present disclosure have been shown and described above, it will be of course understood by those skilled in the art that various modifications may be made without departing from the gist of the disclosure as defined in the following claims, and it is to be noted that those modifications should not be understood individually from the technical concept and prospect of the present disclosure.

1. A method of measuring urine pressure using an image captured by a camera, the method comprising:
acquiring an image capturing a urine stream;
obtaining a measurement variable representing a distance between two points where the urine stream is the thinnest based on the acquired image; and
calculating urine pressure using the obtained measurement variable.

2. The method of claim 1, wherein the acquiring of the image comprises:
checking whether at least one cycle having two points at both ends where the urine stream is the thinnest is present in the image.

3. The method of claim 1, wherein the obtaining of the measurement variable comprises:
detecting a first point and a second point where the urine stream is the thinnest in the acquired image;
calculating a horizontal distance between the first and second points and a drop between the first and second points; and
computing the measurement variable using the calculated horizontal distance and drop.

4. The method of claim 1, wherein the obtaining of the measurement variable comprises:
detecting, in the acquired image, a first point where the urine stream is the thinnest and a third point where the urine stream is the thickest;
calculating a distance between the detected first and third points; and
computing the measurement variable using the calculated distance between the first and third points.

5. The method of claim 1, wherein the calculating of the urine pressure comprises:
computing the urine pressure by multiplying the obtained measurement variable by a proportional constant, a specific gravity of the urine, a unit area, and an acceleration of gravity.

6. The method of claim 1, wherein the acquired image comprises:
a plurality of frames captured at regular intervals from the start of urination to the end of urination.

7. The method of claim 6, further comprising:
displaying at least one of a graph representing a change in urine pressure, a maximum urine pressure value, and an average urine pressure value, using the computed urine pressure for each of the plurality of frames.

8. A urine pressure measurement device, the device comprising:
an image acquisition unit for acquiring an image capturing a urine stream; and
a urine pressure calculation unit for obtaining a measurement variable representing a distance between two points where the urine stream is the thinnest based on the acquired image, and calculating urinary pressure using the obtained measurement variable.

9. The device of claim 8, wherein the image acquisition unit comprises:
a camera mounted at a specific location to capture an image in a direction perpendicular to a direction of the urine stream, and
wherein the camera is mounted at a location spaced apart by a specific distance from a subject so as to allow at least one cycle having two ends at both ends where the urine stream is the thinnest is present in the image.

10. The device of claim 8, wherein the urine pressure calculation unit detects a first point and a second point where the urine stream is the thinnest in the acquired image, calculates a horizontal distance between the first and second points and a drop between the first and second points, and computes the measurement variable using the calculated horizontal distance and drop.

11. The device of claim 8, wherein the pressure calculation unit detects a first point where the urine stream is the thinnest and a third point where the urine stream is the thickest in the acquired image, calculates a distance between the detected first and third points, and computes the measurement variable using the calculated distance between the first and third points.

12. The device of claim 8, wherein the urine pressure calculation unit computes the urine pressure by multiplying the obtained measurement variable by a proportional constant, a specific gravity of the urine, a unit area, and an acceleration of gravity.
